(19) United States
(12) Patent Application Publication
(10) Pub. No.: US 2011/0306622 A1
(43) Pub. Date: Dec. 15, 2011

(54) METHODS OF TREATING HEMATOLOGICAL DISORDERS WITH QUINAZOLINONE COMPOUNDS IN SELECTED SUBJECTS

(75) Inventors: Brian LANNUTTI, Seattle, WA (US); Langdon Miller, Lebanon, NJ (US); Heather Webb, Seattle, WA (US)

(73) Assignee: Calitoga Pharmaceuticals, Inc., Seattle, WA (US)

(21) Appl. No.: 13/158,173

(22) Filed: Jun. 10, 2011

Related U.S. Application Data
(60) Provisional application No. 61/354,152, filed on Jun. 11, 2010.
(60) Provisional application No. 61/415,300, filed on Nov. 18, 2010.

(51) Int. Cl. A61K 31/522; A61P 7/00

(52) U.S. Cl. 514/263.21

(57) ABSTRACT

This disclosure relates to methods of selecting a subset of subjects having a hematological disorder and treating the selected group with a PI3K-delta inhibitor. In particular, the methods disclose evaluating levels of characteristic chemokine biomarkers, such as CCL2, CCL3, CCL4, CCL5, CXCL13, CCL17, CCL22, or TNF-alpha to select subjects that would have a greater chance of benefiting from treatment with a PI3K-delta inhibitor. The PI3K-delta inhibitors disclosed in this application are a type of quinazolinone-purinyl family of compounds.

### CROSS-REFERENCE TO RELATED APPLICATIONS

This application claims the benefit under 35 U.S.C. §119(e) of U.S. provisional patent application Nos. 61/354,152 filed Jun. 11, 2010; and 61/415,300 filed Nov. 18, 2010. The contents of these documents are incorporated herein by reference in their entirety.

### TECHNICAL FIELD

The field of this disclosure is related to methods of selecting a subset population of subjects for treatment with a compound; specifically using biomarkers to select subjects that would benefit from treatment with a PI3K-delta inhibitor, or to select a suitable anticancer drug for a particular subject.

### BACKGROUND ART

Chemokines and cytokines are important markers for some diseases. For example, elevated levels of certain chemokines are important indicators for the severity or the progress of hematological disorders, such as Chronic Lymphocytic Leukemia (CLL), Non-Hodgkin's Lymphoma (NHL), and Mantle Cell Lymphoma (MCL).

It is well established that CLL is a heterogeneous disease: some patients experience a slowly progressive clinical course, but most will eventually enter an advanced phase requiring repeated treatment. A significant number of CLL patients exhibit an active form of the disease from the early stages, characterized by refractoriness to treatment, infectious and autoimmune complications and a relatively rapid fatal outcome.

It is also known that not all cancer patients respond to a particular drug, even though the drug is generally useful for the particular type of cancer. Some individuals respond better to a particular drug or type of treatment than other individuals. The ability to identify a subset population of cancer patients who might benefit from a particular treatment, for example, PI3K-delta inhibitors, would be useful.

There is a need for providing a method of identifying subjects within a population of subjects having a hematological disease who would benefit from treatment with a PI3K-delta inhibitor. This disclosure addresses that need.

### DISCLOSURE OF THE INVENTION

The present disclosure provides methods to select or identify a sub-population of subjects having a hematological disorder for treatment with a PI3K-delta inhibitor.

The methods disclosed herein typically involve detecting the presence of the biomarker chemokine or chemokines in a sample taken from a subject. Not all subjects with a hematological disorder necessarily have elevated chemokine levels. The chemokine levels of subjects with hematological disorders may vary. In some cases, elevated chemokine levels are indicative of a more aggressive form of a disease which may progress more quickly; whereas lower levels of chemokine levels indicate a more stable form of the disease. In some embodiments, subjects with greater elevated chemokine levels than those with lower levels are preferably selected for treatment with a PI3K-delta inhibitor.

In certain embodiments, the level of the biomarker chemokine will be compared to a control value obtained from a normal subject free of a hematological disorder. In other embodiments, the level of the biomarker chemokine will be compared to a control value obtained from a subject having a hematological disorder.

In one embodiment, the disclosure provides a method of treating a hematological disorder in a subject, comprising the steps of a) selecting a subject having an elevated concentration of at least one biomarker selected from the group consisting of CCL2, CCL3, CCL4, CCL5, CXCL13, CCL17, CCL22, and TNF-alpha; and b) administering an effective amount of a PI3K-delta inhibitor to the subject.

In another embodiment, the disclosure provides a method of selecting a subject for treatment, wherein the subject has a hematological disorder, the method comprising the steps a) detecting the level of at least one chemokine in the subject; and b) comparing the chemokine level to levels in a normal subject, wherein an elevated level of chemokine in the subject is indicative of a patient to be selected for treatment with a PI3K-delta inhibitor.

In another embodiment, the disclosure provides a method of selecting a subject for treatment, wherein the subject has a hematological disorder, the method comprising the steps a) detecting the level of at least one chemokine in the subject; and b) comparing the chemokine level to the median for the type of cancer being treated, wherein a chemokine level in the subject above the median for the type of cancer being treated is indicative of a patient to be selected for treatment with a PI3K-delta inhibitor.

In yet another embodiment, the disclosure provides a method to select a chemotherapeutic agent for a patient in need of treatment for a hematological disorder, comprising the steps of determining a level of at least one chemokine selected from CCL2, CCL3, CCL4, CCL5, CXCL13, CCL17, CCL22, and TNF-alpha in the subject's blood or plasma; comparing the level of at least one chemokine in the subject's blood or plasma to a normal level for subjects without the hematological disorder; and if the level of at least one chemokine selected from CCL2, CCL3, CCL4, CCL5, CXCL13, CCL17, CCL22, and TNF-alpha in the subject's blood or plasma is elevated above a normal level for subjects without a hematological disorder, then selecting a PI3K-delta inhibitor to treat the patient. In some embodiments, the elevated level for CCL3 or CCL4 is at least about five fold greater than normal levels, and/or the elevated level for CXCL13 is at least about ten fold greater than normal levels.

In yet another embodiment, the disclosure provides a method of predicting whether a subject with a hematological disorder will respond effectively to treatment with PI3K-delta inhibitor, comprising assessing as a biomarker in sample from the patient the amount of at least one biomarker selected from the group consisting of CCL2, CCL3, CCL4, CCL5, CXCL13, CCL17, CCL22, and TNF-alpha, and predicting the subject will respond effectively to treatment with the inhibitor.

In some of the foregoing embodiments, the PI3K-delta inhibitor is a compound of formula 1:

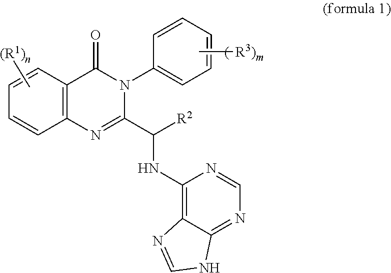

wherein each R¹ is independently selected from the group consisting of halo, CF₃, and C1-C6 alkyl; each R³ is independently selected from the group consisting of halo, CF₃, and C1-C6 alkyl; R² is hydrogen or C1-C6 alkyl; n is an integer from 0 to 2; and m is an integer from 0 to 2, or a pharmaceutically acceptable salt thereof. In some embodiments the compound is the S-enantiomer.

In some of the foregoing embodiments, subjects are relapsed or refractory from other treatment.

### BRIEF DESCRIPTION OF THE DRAWINGS

FIG. 1 shows a series of graphs summarizing the in vivo reduction in elevated CCL3, CCL4 and CXCL13 chemokine levels in CLL patients after one cycle of treatment with Compound A.

FIG. 2 shows a graph summarizing the in vitro reduction in BCR-induced secretion of CCL3 and CCL4 chemokines by CLL cells in the presence of Compound A.

FIG. 3a shows a table summarizing patient characteristics in a study involving 103 patients with indolent non-Hodgkin lymphoma (iNHL) and mantle cell lymphoma (MCL). FIG. 3b shows a table summarizing treatment disposition of the patients in the same study.

FIG. 4a shows a graph depicting the change in tumor size in MCL patients. FIG. 4b shows a graph depicting the change in tumor size in iNHL patients.

FIG. 5 shows a series of graphs summarizing the in vivo reduction in elevated CCL17, CCL22 and CXCL13 chemokine levels, and TNF-alpha levels in MCL and iNHL patients after 28 days of treatment with Compound A.

FIG. 6 shows a graphical summary depicting the levels of TNF-alpha, CXCL13, and CCL22 in patients with hematological malignancies.

FIG. 7 shows a graphical summary depicting the levels of CCL3, CCL4, and CCL2 in patients with hematological malignancies.

FIG. 8 shows a graphical summary depicting the levels of CCL17 in patients with hematological malignancies.

FIG. 9 shows a graphical summary depicting the levels of CXCL12 in patients with hematological malignancies.

FIG. 10 shows a graphical summary (based on mean and 95% Cl) depicting the levels of CCL3, CCL17, and TNF-alpha in CLL patients who responded to treatment with a PI3K-delta inhibitor compared to patients who did not respond to the treatment.

### MODES OF CARRYING OUT THE INVENTION

A “biomarker” is a molecule produced by diseased cells, e.g. by cancer cells, whose expression is useful for identifying a patient who can benefit from therapy with a drug, such as an PI3K-delta inhibitor. Positive expression of the biomarker, as well as increased (or decreased) level relative to cancer cells of the same cancer type or relative to non-diseased cells can be used to identify patients for therapy. Biomarkers described herein include CCL2, CCL3, CCL4, CCL5, CXCL13, CCL17, CCL22, and TNF-alpha. The present application specifically contemplates combining one or more elevated biomarkers to identify patients most likely to respond to therapy with a PI3K-delta inhibitor. The present application also discloses certain levels that are indicative of elevated biomarker concentrations.

Not all subjects with a hematological disorder necessarily have elevated chemokine levels. The chemokine levels of subjects with hematological disorders may vary. Subjects with greater elevated chemokine levels than those with lower levels are preferably selected for treatment with a PI3K-delta inhibitor. In addition, the degree of elevation of the chemokine level may be correlated to the severity or advancement of the disease. The disclosure provides methods to select a sub-population of subjects having a hematological disorder for treatment with a PI3K-delta inhibitor based on elevated biomarker levels in plasma.

The disclosed methods typically involve detecting the presence of an elevated biomarker in a sample taken from a subject. In some embodiments, the level of the biomarker will be compared to a control value obtained from a normal subject free of a hematological disorder. In some embodiments, the level of a chemokine is considered elevated when it is at least twice the normal level, or when it is at least three times the normal level. In some embodiments, at least one elevated biomarker is at least 5-fold greater than subjects free of the hematological disorder. In some embodiments, at least one elevated biomarker is at least 10-fold greater than subjects free of the hematological disorder.

In other embodiments, at least one elevated biomarker is at a level above the median for the type of cancer being treated. In this embodiment, the elevated biomarker is defined “at a level above the median” in which the level of biomarker expression is considered “high expression” to a skilled person for that type of cancer. In one embodiment, the elevated level is greater than 25% above the median. In one embodiment, the elevated level is greater than 50% above the median. In one embodiment, the elevated level is greater than 100% above the median. In one embodiment, the elevated level is greater than 200% above the median. In one embodiment, the elevated level will be in the range from greater than 50% to about 100%, e.g. from about 75% to about 100% relative to biomarker level in a population of samples, cells, tumors, or cancers of the same cancer type. In one embodiment, e.g. for the biomarkers described herein, such high expression will be at least one standard deviation above the median. Such “high expressing” samples may express the biomarker at a 2+ or 3+ level.

High blood plasma levels of certain chemokines may define a characteristic that may be associated with aggressive disease. In particular, patients having chronic lymphocytic leukemia (CLL) may exhibit elevated levels of CCL2, CCL3, CCL4, CCL5, CXCL13, CCL17, CCL22, and TNF-alpha, or combinations thereof. In some embodiments, patients having chronic lymphocytic leukemia (CLL) may exhibit elevated levels of CCL3, CCL4, CXCL13 or combinations thereof. In other embodiments, patients having chronic lymphocytic leukemia (CLL) may exhibit elevated levels of CCL2, CCL3, CCL4, or combinations thereof. In yet other embodiments, patients having chronic lymphocytic leukemia (CLL) may exhibit elevated levels of CCL2, CCL3, CCL4, CXCL13 or combinations thereof.

In some embodiments, patients having Hodgkin's lymphoma may exhibit elevated levels of CCL3, CCL4, CCL5, CXCL13, CCL17, and CCL22, or combinations thereof. In some embodiments, patients having Hodgkin's lymphoma may exhibit elevated levels of CCL5, CCL17, CCL22, or combinations thereof. In some embodiments, patients having non-Hodgkin's lymphoma (NHL) or mantle cell lymphoma (MCL) may exhibit elevated levels of CCL17, CCL22, CXCL13, and TNF-alpha, or combinations thereof. In some embodiments, patients having non-Hodgkin's lymphoma (NHL) may exhibit elevated levels of CCL17.

In one embodiment, the disclosure provides a method of treating a hematological disorder in a subject, comprising the steps of a) selecting a subject having elevated chemokine concentration of at least one chemokine selected from the group consisting of CCL3, CCL4, CCL5, CXCL13, CCL17 and CCL22; and b) administering an effective amount of a PI3K-delta inhibitor to the subject.

In another embodiment, the disclosure provides a method of treating a hematological disorder in a subject, comprising the steps of a) selecting a subject having an elevated concentration of at least one biomarker selected from the group consisting of CCL2, CCL3, CCL4, CCL5, CXCL13, CCL17, CCL22, and TNF-alpha; and b) administering an effective amount of a PI3K-delta inhibitor to the subject.

In another embodiment, the disclosure provides a method of selecting a subject for treatment, wherein the subject has a hematological disorder, the method comprising the steps a) detecting the level of at least one chemokine in the subject; and b) comparing the chemokine level to levels in a normal subject, wherein an elevated level of chemokine in the subject is indicative of a patient to be selected for treatment with a PI3K-delta inhibitor.

In yet another embodiment, this application discloses a method to select a chemotherapeutic agent for a patient in need of treatment for a hematological disorder, comprising the steps of determining a level of at least one chemokine selected from CCL3, CCL4, CCL5, CXCL13, CCL17 and CCL22 in the subject's blood or plasma; comparing the level of at least one chemokine in the subject's blood or plasma to a normal level for subjects without the hematological disorder; and if the level of at least one chemokine selected from CCL3, CCL4, and CXCL13 in the subject's blood or plasma is elevated above a normal level for subjects without a hematological disorder, then selecting a PI3K-delta inhibitor to treat the patient.

In yet another embodiment, this application discloses a method of predicting whether a subject with a hematological disorder will respond effectively to treatment with PI3K-delta inhibitor, comprising assessing as a biomarker in sample from the patient the amount of at least one biomarker selected from the group consisting of CCL2, CCL3, CCL4, CCL5, CXCL13, CCL17, CCL22, and TNF-alpha, and predicting the subject will respond effectively to treatment with the inhibitor.

In some embodiments, the concentration of at least one chemokine is decreased by at least 2-fold after administration of a PI3K-delta inhibitor. In other embodiments, the concentration of at least one chemokine is decreased by at least 3-fold after administration of a PI3K-delta inhibitor

The elevated level for CCL3 or CCL4 can be at least about five fold greater than normal levels, and the elevated level for CXCL13 can be at least about ten fold greater than normal levels.

In one embodiment, the subject has elevated chemokine levels for at least two of the chemokines CCL2, CCL3, CCL4, CCL5, CXCL13, CCL17, CCL22, and TNF-alpha. In another embodiment, the subject has elevated chemokine levels for at least two of the chemokines CCL3, CCL4, CCL5, CXCL13, CCL17 and CCL22. In another embodiment, the subject has elevated chemokine levels for chemokines CCL3, CCL4, and CXCL13. In another embodiment, the subject has elevated chemokine levels for chemokines CCL2, CCL3, and CCL4. In another embodiment, the subject has elevated chemokine levels for chemokine CCL17.

In some of the foregoing embodiments, the subject has elevated levels of CLL2, CCL3, CCL4, or combinations thereof, and the elevated levels are at least about 5-fold greater compared to subjects free of the hematological disorder. In some of the foregoing embodiments, the subject has elevated levels of CCL3 or CCL4, or both CCL3 and CCL4, and the elevated levels are at least about 5-fold greater compared to subjects free of the hematological disorder. In some of the foregoing embodiments, the subject has elevated levels of CLL2, CCL3, CCL4, CXCL13 or combinations thereof, and the elevated levels are at least about 5-fold greater compared to subjects free of the hematological disorder. In some of the foregoing embodiments, the subject has elevated levels of CCL2 at least about 5-fold greater compared to subjects free of the hematological disorder. In some of the foregoing embodiments, the subject has elevated levels of CCL3 at least about 5-fold greater compared to subjects free of the hematological disorder. In some of the foregoing embodiments, the subject has elevated levels of CCL4 at least about 5-fold greater compared to subjects free of the hematological disorder. In some of the foregoing embodiments, the subject has elevated levels of CXCL13 at least about 5-fold greater compared to subjects free of the hematological disorder. In some of the foregoing embodiments, the subject has elevated levels of CCL3 and CCL4 at least about 5-fold greater compared to subjects free of the hematological disorder. In some of the foregoing embodiments, the subject has elevated levels of CCL2, CCL3 and CCL4 at least about 5-fold greater compared to subjects free of the hematological disorder. In some of the foregoing embodiments, the subject has elevated levels of CCL2, CCL3, CCL4, and CXCL13 at least about 5-fold greater compared to subjects free of the hematological disorder.

In some of the foregoing embodiments, the subject has an elevated level of at least CXCL13, and the elevated level of CXCL13 is at least about 10-fold greater compared to subjects free of the hematological disorder. In some of the foregoing embodiments, the subject has elevated levels of CCL17 at least about 5-fold greater compared to subjects free of the hematological disorder and an elevated level of CCL17 at least about 10-fold greater compared to subjects free of the hematological disorder.

In some of the foregoing embodiments, the subject has elevated levels of CCL17, CCL22, CXCL13, TNF-alpha, or combinations thereof, and the elevated levels are at least about 5-fold greater compared to subjects free of the hematological disorder. In some of the foregoing embodiments, the subject has elevated levels of CCL17 at least about 5-fold greater compared to subjects free of the hematological disorder. In some of the foregoing embodiments, the subject has elevated levels of CCL22 at least about 5-fold greater compared to subjects free of the hematological disorder. In some of the foregoing embodiments, the subject has elevated levels of CXCL13 at least about 5-fold greater compared to subjects free of the hematological disorder. In some of the foregoing embodiments, the subject has elevated levels of TNF-alpha at least about 5-fold greater compared to subjects free of the hematological disorder. In some of the foregoing embodiments, the subject has elevated levels of CCL17, CCL22, CXCL13, and TNF-alpha at least about 5-fold greater compared to subjects free of the hematological disorder.

In some embodiments, the subject is suitable for treatment with a PI3K-delta inhibitor as described herein when at least one of the subject's chemokine levels is elevated above a specific cut-off level. Suitable cut-off levels for use in such methods include 700, 750, 800 pg/mL for CCL2; 100, 150, or 200 pg/mL for CCL3; 150, 200, 250 or 300 pg/mL for CCL4; 150, 200 or 250 pg/mL for CXCL13; 25, 50, 75 pg/mL for TNF-alpha; 700, 750, 800 pg/mL for CCL17; and 1750, 2000, 2250 pg/mL for CCL22.

In some of the foregoing embodiments, the subject has elevated levels of at least CCL2, and the average plasma concentration of CCL2 is at least about 750 pg/mL. In some of the foregoing embodiments, the subject has elevated levels of at least CCL2, and the average plasma concentration of CCL2 is at least about 700 pg/mL. In some of the foregoing embodiments, the subject has elevated levels of at least CCL2, and the average plasma concentration of CCL2 is at least about 650 pg/mL. In some of the foregoing embodiments, the subject has elevated levels of at least CCL2, and the average plasma concentration of CCL2 is at least about 800 pg/mL.

In some of the foregoing embodiments, the subject has elevated levels of at least CCL3, and the average plasma concentration of CCL3 is at least about 150 pg/mL. In some of the foregoing embodiments, the subject has elevated levels of at least CCL3, and the average plasma concentration of CCL3 is at least about 100 pg/mL. In some of the foregoing embodiments, the subject has elevated levels of at least CCL3, and the average plasma concentration of CCL3 is at least about 75 pg/mL. In some of the foregoing embodiments, the subject has elevated levels of at least CCL3, and the average plasma concentration of CCL3 is at least about 200 pg/mL.

In some of the foregoing embodiments, the subject has elevated levels of at least CCL4, and the average plasma concentration of CCL4 is at least about 250 pg/mL. In some of the foregoing embodiments, the subject has elevated levels of at least CCL4, and the average plasma concentration of CCL4 is at least about 200 pg/mL. In some of the foregoing embodiments, the subject has elevated levels of at least CCL4, and the average plasma concentration of CCL4 is at least about 150 pg/mL. In some of the foregoing embodiments, the subject has elevated levels of at least CCL4, and the average plasma concentration of CCL4 is at least about 300 pg/mL.

In some of the foregoing embodiments, the subject has elevated levels of at least CXCL13, and the average plasma concentration of CXCL13 is at least about 200 pg/mL. In some of the foregoing embodiments, the subject has elevated levels of at least CXCL13, and the average plasma concentration of CXCL13 is at least about 175 pg/mL. In some of the foregoing embodiments, the subject has elevated levels of at least CXCL13, and the average plasma concentration of CXCL13 is at least about 150 pg/mL. In some of the foregoing embodiments, the subject has elevated levels of at least CXCL13, and the average plasma concentration of CXCL13 is at least about 250 pg/mL.

In some of the foregoing embodiments, the subject has elevated levels of at least CCL17, and the average plasma concentration of CCL17 is at least about 750 pg/mL. In some of the foregoing embodiments, the subject has elevated levels of at least CCL17, and the average plasma concentration of CCL17 is at least about 700 pg/mL. In some of the foregoing embodiments, the subject has elevated levels of at least CCL17, and the average plasma concentration of CCL17 is at least about 650 pg/mL. In some of the foregoing embodiments, the subject has elevated levels of at least CCL17, and the average plasma concentration of CCL17 is at least about 800 pg/mL.

In some of the foregoing embodiments, the subject has elevated levels of at least CCL22, and the average plasma concentration of CCL22 is at least about 2000 pg/mL. In some of the foregoing embodiments, the subject has elevated levels of at least CCL22, and the average plasma concentration of CCL22 is at least about 1750 pg/mL. In some of the foregoing embodiments, the subject has elevated levels of at least CCL22, and the average plasma concentration of CCL22 is at least about 1500 pg/mL. In some of the foregoing embodiments, the subject has elevated levels of at least CCL22, and the average plasma concentration of CCL22 is at least about 2250 pg/mL.

In some of the foregoing embodiments, the subject has elevated levels of at least TNF-alpha, and the average plasma concentration of TNF-alpha is at least about 50 pg/mL. In some of the foregoing embodiments, the subject has elevated levels of at least TNF-alpha, and the average plasma concentration of TNF-alpha is at least about 40 pg/mL. In some of the foregoing embodiments, the subject has elevated levels of at least TNF-alpha, and the average plasma concentration of TNF-alpha is at least about 25 pg/mL. In some of the foregoing embodiments, the subject has elevated levels of at least TNF-alpha, and the average plasma concentration of TNF-alpha is at least about 55 pg/mL.

In some of the foregoing embodiments, the subject has elevated levels of CCL3, CCL4 and CXCL13; wherein the average plasma concentration of CCL3 is at least about 150 pg/mL; the average plasma concentration of CCL4 is at least about 250 pg/mL and the average plasma concentration of CXCL13 is at least about 200 pg/mL.

In some of the foregoing embodiments, the subject has elevated levels of CCL2, CCL3, and CCL4; wherein the average plasma concentration of CLL2 is at least about 750 pg/mL; the average plasma concentration of CCL3 is at least about 150 pg/mL; and the average plasma concentration of CCL4 is at least about 250 pg/mL.

In some of the foregoing embodiments, the method comprises detecting the level of said chemokine(s) in a fluid or tissue of the subject. In some of the foregoing embodiments, the method comprises detecting the level of said chemokine(s) in plasma of the subject. In some of the foregoing embodiments, the method comprises detecting the level of said chemokine(s) in blood of the subject.

In some of the foregoing embodiments, the concentration of at least one chemokine is decreased by at about 2 to 5-fold after BID administration of a PI3K-delta inhibitor over a duration of 1 week.

The phrase “chemokine biomarker” or “chemokine marker” as used herein refers to the following chemokines: CCL2, CCL3, CCL4, CCL5, CXCL13, CCL17, CCL22 and TNF-alpha. In some instances, the phrase “chemokine biomarker” or “chemokine marker” refers to a polypeptide fragment of CCL2, CCL3, CCL4, CCL5, CXCL13, CCL17, CCL22 and TNF-alpha. “Diagnostic levels” of chemokine biomarkers as used herein refer to the presence of levels of CCL2, CCL3, CCL4, CCL5, CXCL13, CCL17, CCL22 and TNF-alpha that are statistically significantly elevated relative to a normal subject or elevated relative to a level above the median for the type of cancer being treated.

The term “immunoassay” refers to an assay that uses an antibody or antibodies to specifically bind an antigen. The immunoassay is characterized by the use of specific binding properties of a particular antibody or antibodies to isolate, target, and/or quantify the antigen.

“Specific binding” between a binding agent, e.g., a protein, for instance, a biomarker chemokine, refers to the ability of a capture or detection-agent to preferentially bind to a particular chemokine that is present in a mixture; e.g., blood plasma. In some embodiments, specific binding means a dissociation constant (KD) that is less than about 10⁻⁶ M. In some embodiments, specific binding means a dissociation constant (KD) that is less than about 10⁻⁸M. In some embodiments, specific binding means a dissociation constant (KD) that is less than about 10⁻⁹ M.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample.

The phrase “level of chemokine biomarker” in a biological sample as used herein typically refers to the amount of protein, protein fragment or peptide levels of the chemokine biomarker (for example, CCL2, CCL3, CCL4, CCL5, CXCL13, CCL17, CCL22 and TNF-alpha) that is present in a biological sample. In some embodiments, a “level of chemokine biomarker” need not be quantified, but can simply be detected, e.g., a subjective, visual detection by a human, with or without comparison to a level from a control sample or a level expected of a control sample.

The PI3K-delta inhibitor administered to a subject according to this disclosure is a compound of formula 1:

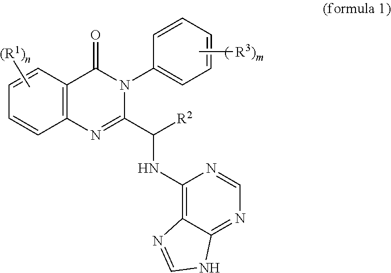

wherein each R¹ is independently selected from the group consisting of halo, CF₃, and C1-C6 alkyl; each R³ is independently selected from the group consisting of halo, CF₃, and C1-C6 alkyl; R² is hydrogen or C1-C6 alkyl; n is an integer from 0 to 2; and m is an integer from 0 to 2, or pharmaceutically acceptable salts thereof.

In some of the foregoing embodiments, each R¹ is independently selected from the group consisting of F, Cl, Br, methyl, ethyl and propyl. In some of the foregoing embodiments, each R¹ is F, Cl or methyl. In some of the foregoing embodiments, each R³ is independently selected from the group consisting of F, Cl, Br, methyl, ethyl and propyl. In some of the foregoing embodiments, each R³ is independently F or methyl. In some of the foregoing embodiments, R² is hydrogen, methyl, ethyl or propyl.

In some of the foregoing embodiments, n is 1; R¹ is F, Cl or methyl; R² is methyl or ethyl; m is 0, 1, or 2; and R³ is F. In some of the foregoing embodiments, n is 1; R¹ is F; R² is ethyl; and m is 0. In some of the foregoing embodiments, n is 1; R¹ is F; R² is methyl; and m is 0. In some of the foregoing embodiments, n is 1; R¹ is Cl; R² is methyl; and m is 0. In some of the foregoing embodiments, n is 0; R² is methyl; m is 2; and R³ is F. In some of the foregoing embodiments, n is 0; m is 0, and R² is methyl or ethyl. In some of the foregoing embodiments, n is 1; R¹ is F; R² is methyl; m is 2; and R³ is F. In some of the foregoing embodiments, n is 1; R¹ is Cl; R² is ethyl; m is 2; and R³ is F.

In some of the foregoing embodiments, R² is C1-C6 alkyl, wherein the resulting stereocenter forms the S-enantiomer:

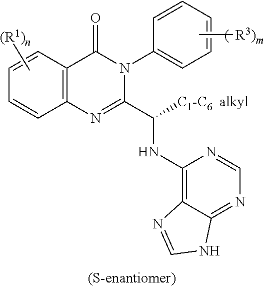

In some embodiments the compound is selected from the group consisting of 2-(1-(9H-purin-6-ylamino)propyl)-5-fluoro-3-phenylquinazolin-4(3H)-one; 2-(1-(9H-purin-6-ylamino)ethyl)-6-fluoro-3-phenylquinazolin-4(3H)-one; and 2-(1-(9H-purin-6-ylamino)ethyl)-3-(2,6-difluorophenyl)quinazolin-4(3H)-one or a pharmaceutically acceptable salt thereof. In more specific embodiments the compound is the S-enantiomer, or a pharmaceutically acceptable salt thereof. For instance, the compound S-enantiomer of 2-(1-(9H-purin-6-ylamino)propyl)-5-fluoro-3-phenylquinazolin-4(3H)-one is shown below as Compound A:

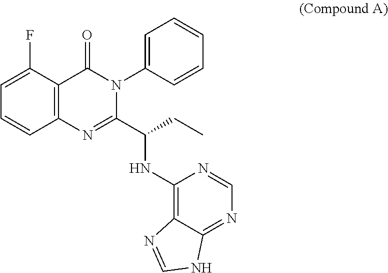

In some embodiments, the dosage of the PI3K-delta inhibitor is selected to reduce the elevated chemokine level to that of or near a normal level of a subject free of the hematological disorder. Typically, the dosage of the PI3K-delta inhibitor is sufficient to reduce the elevated chemokine levels below the threshold levels described herein for identifying the subject as suitable for treatment with a PI3K-delta inhibitor. Other factors that would guide the amount of compound include undesirable side-effects as a result of a administering certain dosage. In this case, the dosage would be reduced to an amount that would not cause the undesirable side-effect.

In some of the foregoing embodiments, the dose of the PI3K-delta inhibitor administered to a subject selected by the methods disclosed herein is about 50 to 350 mg BID. In some of the foregoing embodiments, the concentration of at least one chemokine is decreased by at least 2-fold after administration of a PI3K-delta inhibitor at a dose about 50 to 350 mg BID. In some of the foregoing embodiments, the dose of compound A administered to a subject selected by the methods disclosed herein is about 50 to 350 mg BID. In some of the foregoing embodiments, the dose of compound A administered to a subject selected by the methods disclosed herein is at least about 50 mg BID. In some of the foregoing embodiments, the dose of compound A administered to a subject selected by the methods disclosed herein is at least about 100 mg BID. In some of the foregoing embodiments, the dose of compound A administered to a subject selected by the methods disclosed herein is at least about 200 mg BID. In some of the foregoing embodiments, the dose of compound A administered to a subject selected by the methods disclosed herein is at least about 250 mg BID. In some of the foregoing embodiments, the dose of compound A administered to a subject selected by the methods disclosed herein is at least about 300 mg BID.

In some embodiments, the PI3K-delta inhibitor is selective for PI3K-delta. The term “selective PI3Kδ inhibitor”, etc., as used herein, refers to a compound that inhibits the PI3Kδ isozyme more effectively than at least one other isozymes of the PI3K family. The selective inhibitor may also be active against other isozymes of PI3K, but requires higher concentrations to achieve the same degree of inhibition of the other isozymes. “Selective” can also be used to describe a compound that inhibits a particular PI3-kinase more so than a comparable compound. A “selective PI3Kδ inhibitor” compound is understood to be more selective for PI3Kδ than compounds conventionally and generically designated PI3K inhibitors, e.g., wortmannin or LY294002. Concomitantly, wortmannin and LY294002 are deemed “nonselective PI3K inhibitors.” Typically the selective inhibitor is at least about 10-fold more potent (lower IC-50) on PI3K-delta than on the alpha, beta and/or delta isoforms. Compound A is an example of a selective PI3K-delta inhibitor.

In certain embodiments, compounds of any type that selectively negatively regulate PI3Kδ expression or activity can be used as selective PI3Kδ inhibitors in the methods of this disclosure. Moreover, compounds of any type that selectively negatively regulate PI3Kδ expression or activity and that possess acceptable pharmacological properties can be used as selective PI3Kδ inhibitors in the therapeutic methods of this disclosure. Without being bound by theory, targeting p110 delta inhibition with a compound of this disclosure provides an effective approach for the treatment of hematological malignancies because this method inhibits constitutive signaling resulting in direct destruction of the tumor cell. In addition, without being bound by theory, p110 delta inhibition represses microenvironmental signals which are crucial for tumor cell homing, survival and proliferation.

In specific embodiments, this application discloses a method of treating CLL in a subject, comprising the steps of a) selecting a subject having elevated chemokine concentration of at least one chemokine selected from the group consisting of CCL3, CCL4, and CXCL13; and b) administering an effective amount of a PI3K-delta selective inhibitor to the subject, wherein the PI3K-delta selective inhibitor, which can be Compound A; wherein an elevated level of CCL3 or CCL4 is at least about 5-fold greater compared to subjects free of the hematological disorder, and/or wherein an elevated level of CXCL13 is at least about 10-fold greater compared to subjects free of the hematological disorder.

In specific embodiments, this disclosure provides a method of selecting a subject for treatment with a PI3K-delta inhibitor, preferably a selective PI3K-delta inhibitor, wherein the subject has CLL, the method comprising the steps a) detecting the level of at least one chemokine in the subject selected from the group consisting of CCL3, CCL4 and CXCL13; and b) comparing the chemokine level to levels in a normal subject, wherein an elevated level of chemokine in the subject is indicative of a patient to be selected for treatment with a PI3K-delta selective inhibitor; wherein an elevated level of CCL3 or CCL4 is at least about 5-fold greater compared to subjects free of the hematological disorder, and wherein an elevated level of CXCL13 is at least about 10-fold greater compared to subjects free of the hematological disorder. Alternatively, the subject can be suitable for treatment if one or two or all three of these chemokine levels are above a specific cut-off level as disclosed herein. In some such embodiments, the subject is selected for such treatment if two of the three chemokine levels are so elevated. In some embodiments, the subject is selected for such treatment if all three of the chemokine levels are elevated.

In specific embodiments, this disclosure provides a method to select a chemotherapeutic agent for a patient in need of treatment for CLL, comprising the steps of determining a level of at least one chemokine selected from CCL3, CCL4, and CXCL13 in the subject's blood or plasma; comparing the level of at least one chemokine in the subject's blood or plasma to a normal level for subjects without the hematological disorder, or to a specific cut-off value; and if the level of at least one chemokine selected from CCL3, CCL4, and CXCL13 in the subject's blood or plasma is elevated above a normal level for subjects without a hematological disorder, or above a specific cut-off value, then selecting a PI3K-delta selective inhibitor to treat the patient; wherein an elevated level for CCL3 or CCL4 is at least about five fold greater than normal levels and an elevated level for CXCL13 is at least about ten fold greater than normal levels. Here, too, the selective PI3K-delta inhibitor can be Compound A.

### Patient Population

For the purposes of the application, subjects are individuals who have a hematological disorder. In some of the foregoing embodiments, the hematological disorder is selected from the group consisting of acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), Hodgkin's lymphoma, mantle cell lymphoma (MCL), follicular lymphoma, Waldenstrom's macroglobulinemia (WM), B-cell lymphoma and diffuse large B-cell lymphoma (DLBCL). NHL may include indolent Non-Hodgkin's Lymphoma (iNHL) or aggressive Non-Hodgkin's Lymphoma (aNHL). In some embodiments, subjects are relapsed or refractory from other treatment.

In some embodiments, the hematological disorder is chronic lymphocytic leukemia (CLL) in the method of predicting whether a subject with a hematological disorder will respond effectively to treatment with PI3K-delta inhibitor. In some instances where the hematological disorder is CLL, at least one elevated biomarker is selected from the group consisting of CCL2, CCL3, and CCL4. In particular embodiments, the biomarker concentration of CCL2 is greater than 750 pg/mL, CCL3 is greater than 150 pg/mL, and CCL4 is greater than 250 pg/mL, or a combination of these amounts. In particular embodiments, the biomarker concentration of CCL2 is greater than 800 pg/mL, CCL3 is greater than 200 pg/mL, and CCL4 is greater than 300 pg/mL, or a combination of these amounts. In particular embodiments, the biomarker concentration of CCL2 is greater than 700 pg/mL, CCL3 is greater than 100 pg/mL, and CCL4 is greater than 200 pg/mL, or a combination of these amounts.

In some instances where the hematological disorder is CLL, at least one elevated biomarker is selected from the group consisting of CCL2, CCL3, CCL4, CXCL13, and TNF-alpha. In particular embodiments, the biomarker concentration of CCL2 is greater than 750 pg/mL, CCL3 is greater than 150 pg/mL, CCL4 is greater than 250 pg/mL, CXCL13 is greater than 200 pg/mL, or TNF-alpha is greater than 50 pg/mL, or a combination of these amounts. In particular embodiments, the biomarker concentration of CCL2 is greater than 800 pg/mL, CCL3 is greater than 200 pg/mL, CCL4 is greater than 300 pg/mL, CXCL13 is greater than 250 pg/mL, or TNF-alpha is greater than 75 pg/mL, or a combination of these amounts. In particular embodiments, the biomarker concentration of CCL2 is greater than 700 pg/mL, CCL3 is greater than 100 pg/mL, CCL4 is greater than 200 pg/mL, CXCL13 is greater than 150 pg/mL, or TNF-alpha is greater than 25 pg/mL, or a combination of these amounts.

In certain embodiments where the hematological disorder is CLL, the elevated biomarker concentration of CCL2 is greater than 800 pg/mL, 850 pg/mL, 900 pg/mL, 700 pg/mL, 650 pg/mL, or 600 pg/mL. In certain embodiments that may be combined with any of the preceding embodiments, the elevated biomarker concentration of CCL3 is greater than 200 pg/mL, 250 pg/mL. 300 pg/mL. 100 pg/mL, 75 pg/mL, or 50 pg/mL. In certain embodiments that may be combined with any of the preceding embodiments, the elevated biomarker concentration of CCL4 is greater than 200 pg/mL, 150 pg/mL, 100 pg/mL, 300 pg/mL, 350 pg/mL, or 400 pg/mL. In certain embodiments that may be combined with any of the preceding embodiments, the elevated biomarker concentration of CXCL13 is greater than 150 pg/mL, 100 pg/mL, 75 pg/mL, 250 pg/mL, 300 pg/mL, or 350 pg/mL. In certain embodiments that may be combined with any of the preceding embodiments, the elevated biomarker concentration of TNF-alpha is greater than 45 pg/mL, 40 pg/mL, 25 pg/mL, 55 pg/mL, 60 pg/mL, or 75 pg/mL.

In some embodiments, the hematological disorder is mantle cell lymphoma (MCL) or non-Hodgkin's lymphoma (NHL). In some instances where the hematological disorder is MCL or NHL, at least one elevated biomarker is selected from the group consisting of CCL17, CCL22, CXCL13, and TNF-alpha. In particular embodiments, the biomarker concentration of CCL17 is greater than 150 pg/mL, CCL22 is greater than 2000 pg/mL, CXCL13 is greater than 400 pg/mL, or TNF-alpha is greater than 30 pg/mL, or a combination of these amounts. In particular embodiments, the biomarker concentration of CCL17 is greater than 200 pg/mL, CCL22 is greater than 2250 pg/mL, CXCL13 is greater than 450 pg/mL, or TNF-alpha is greater than 40 pg/mL, or a combination of these amounts. In particular embodiments, the biomarker concentration of CCL17 is greater than 100 pg/mL, CCL22 is greater than 1750 pg/mL, CXCL13 is greater than 350 pg/mL, or TNF-alpha is greater than 25 pg/mL, or a combination of these amounts.

In certain embodiments where the hematological disorder is MCL or NHL, the elevated biomarker concentration of CCL17 is greater than 200 pg/mL, 250 pg/mL, 300 pg/mL, 125 pg/mL, 100 pg/mL, or 75 pg/mL. In certain embodiments that may be combined with any of the preceding embodiments, the elevated biomarker concentration of CCL22 is greater than 2250 pg/mL, 2500 pg/mL, 3000 pg/mL, 1750 pg/mL, 1500 pg/mL, 1250 pg/mL, or 1000 pg/mL. In certain embodiments that may be combined with any of the preceding embodiments, the elevated biomarker concentration of CXCL13 is greater than 450 pg/mL, 500 pg/mL, 550 pg/mL, 350 pg/mL, 300 pg/mL, or 250 pg/mL. In certain embodiments that may be combined with any of the preceding embodiments, the elevated biomarker concentration of TNF-alpha is greater than 25 pg/mL, 20 pg/mL, 15 pg/mL, 35 pg/mL, 40 pg/mL, or 45 pg/mL.

In some embodiments, the hematological disorder is NHL and the elevated biomarker is CCL17. In particular embodiments, the biomarker concentration of CCL17 is greater than 750 pg/mL. In other embodiments, the biomarker concentration of CCL17 is greater than 700 pg/mL, 650 pg/mL, 600 pg/mL, 800 pg/mL, 850 pg/mL, or 900 pg/mL.

In some of the foregoing embodiments, subjects are relapsed or refractory from other treatment. In some of the foregoing embodiments, subjects are relapsed or refractory from at least 2 or more other treatments. In some of the foregoing embodiments, subjects are relapsed or refractory from at least 3 or more other treatments. In some of the foregoing embodiments, subjects are relapsed or refractory from at least 5 or more other treatments.

### Detecting Chemokine Biomarkers

Any one or more of the specified chemokine biomarkers (e.g., CCL2, CCL3, CCL4, CCL5, CXCL13, CCL17, CCL22 and TNF-alpha) can be used to practice the methods of the present disclosure. In some instances, CCL3 alone is used as a diagnostic marker. In some instances, CCL4 alone is used as a diagnostic biomarker. In some other instances, CXCL13 alone is used as a diagnostic marker. In other embodiments, the level of any one or more of the other chemokine biomarkers (CCL2, CCL3, CCL4, CCL5, CXCL13, CCL17, CCL22 and TNF-alpha) are used as diagnostic biomarkers. In other embodiments, specific combinations of biomarkers are used.

The presence or levels of any one or any combination of the chemokine biomarkers CCL2, CCL3, CCL4, CCL5, CXCL13, CCL17, CCL22 and TNF-alpha can be used to select a patient with a hematological disorder for treatment. In some instances, specific combinations of the chemokine biomarkers, such as CCL3, CCL4, and CXCL13, can be used to diagnose the severity of the hematological disorder. In some embodiments, the presence or level of these chemokine biomarkers can be used to select a patient as candidate for treatment. In some other embodiments, the presence or levels of the chemokine markers can be used to determine the success during the course of or after treatment of a subject having a hematological disorder.

In addition to using immunoassays to detect the levels of chemokines in a fluid sample from a subject, assessment of chemokine expression and levels can be made based on the level of gene expression of the particular chemokines. RNA hybridization techniques for determining the presence and/or level of mRNA expression are well known to those of skill in the art and can be used to assess the presence or level of gene expression of the chemokine biomarkers of interest.

### Antibodies and Immunoassays

In some embodiments, the methods and kits of the present disclosure utilize selective binding partners of the chemokine biomarkers to identify the presence or determine the levels of the chemokine biomarkers in a biological sample. The selective binding partner to be used with the methods and kits of the present disclosure can be, for instance, an antibody. In some embodiments, monoclonal antibodies to the particular chemokine biomarkers can be used. In some other embodiments, polyclonal antibodies to the particular chemokine biomarkers can be employed to practice the methods and in the kits of the present disclosure.

Immunoassays can be used to qualitatively or quantitatively analyze the cytokine biomarker levels in a biological sample. A general overview of the applicable technology can be found in a number of readily available manuals, e.g., Harlow & Lane, Cold Spring Harbor Laboratory Press, Using Antibodies: A Laboratory Manual (1999).

Commercial antibodies to the chemokine biomarkers of this disclosure are available and can be used with the methods and kits of the present disclosure.

In some embodiments, the antibodies to be used for the assays of the present disclosure can be produced using techniques for producing monoclonal or polyclonal antibodies that are well known in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Such antibodies can be used for therapeutic and diagnostic applications, e.g., in the treatment and/or detection of any of the specific chemokine-associated diseases or conditions described herein.

A number of a particular cytokine comprising immunogens may be used to produce antibodies specifically reactive with that particular cytokine biomarker. For example, a recombinant CCL2, CCL3, CCL4, CCL5, CXCL13, CCL17, CCL22, or TNF-alpha, or an antigenic fragment thereof, can be isolated using methods well known to those of skill in the art. Recombinant protein can be expressed in eukaryotic or prokaryotic cells. Recombinant protein is the typically used immunogen for the production of monoclonal or polyclonal antibodies. Alternatively, a synthetic peptide derived from the known sequences of the cytokine biomarkers and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Once specific antibodies are available, each specific cytokine biomarker can be detected by a variety of immunoassay methods. For a review of immunological and immunoassay procedures, see Basic and Clinical Immunology (Stites & Ten eds., 7th ed. 1991). Moreover, the immunoassays of the present disclosure can be performed in any of several configurations, which are reviewed extensively in Enzyme Immunoassay (Maggio, ed., 1980); and Harlow & Lane, supra.

Immunological binding assays (or immunoassays) typically use an antibody that specifically binds to a protein or antigen of choice. As described above, the antibody may be produced by any of a number of means well known to those of skill in the art and as described above.

Specific binding of a cytokine to an antibody may typically require an antibody that is selected for its specificity for a particular protein. For example, polyclonal antibodies raised to a particular cytokine can be selected to obtain only those polyclonal antibodies that are specifically immunoreactive with a particular cytokine and not with other proteins, except for polymorphic variants, orthologs, and alleles of the particular cytokine. This selection may be achieved by subtracting out antibodies which react with the cytokine of interest. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically the signal of a specific or selective reaction will be at least twice background signal or noise and more typically more than 10 to 100 times background. Antibodies that react only with a particular cytokine ortholog, e.g., from specific species such as rat, mouse, or human, can also be detected as described above, by subtracting out antibodies that bind to the same cytokine from another species.

Immunoassays also often use a labeling agent to specifically bind to and allow for the detection of the complex formed by the antibody and antigen. The labeling agent may itself be one of the moieties comprising the antibody/antigen complex. Thus, the labeling agent may be a labeled, for instance, anti-CCL2, anti-CCL3, anti-CCL4, anti-CXCL13, anti-CCL5, anti-CCL17, anti-CCL22 antibody or anti-TNF-alpha. Alternatively, the labeling agent may be a third moiety, such a secondary antibody, which specifically binds to the antibody/cytokine complex (a secondary antibody is typically specific to antibodies of the species from which the first antibody is derived). The labeling agent can be modified with a detectable moiety, such as biotin, to which another molecule can specifically bind, such as streptavidin. A variety of detectable moieties are well known to those skilled in the art.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C. In some embodiments, the immunological assay is instantaneous and a read-out for the presence or levels of the cytokine biomarkers is available nearly immediately upon extracting the sample from the subject and performing the immunoassay.

Immunoassays for detecting the cytokine biomarkers in samples may be either competitive or noncompetitive.

Noncompetitive immunoassays are assays in which the amount of antigen is directly measured. In one preferred “sandwich” assay, the anti-CCL2, anti-CCL3, anti-CCL4, anti-CCL5, anti-CXCL13, anti-CCL17, anti-CCL22, or anti-TNF-alpha antibodies, for instance, can be bound directly to a solid substrate on which they are immobilized. These immobilized antibodies then capture the corresponding cytokine present in the test sample. The cytokine is thus immobilized is then bound by a labeling agent, such as a second antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

In competitive assays, the amount of cytokine biomarker present in the sample is measured indirectly by measuring the amount of a known, added (exogenous) cytokine displaced (competed away) from an anti-chemokine antibody by the unknown chemokine present in a sample. A hapten inhibition assay is another competitive assay.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

One of skill in the art will appreciate that it is often desirable to reduce non-specific binding in immunoassays. Particularly, where the assay involves an antigen or antibody immobilized on a solid substrate it is desirable to reduce the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred. In addition to, or in place of proteinaceous material, various detergents can be incorporated into the immunoassay to reduce non-specific interactions.

The particular label or detectable group used in the assay is not a critical aspect of the disclosed methods, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present methods. Thus, a label is any composition detectable by spectroscopic, photochemical, biochemical, immunochemical, radiographic, electrical, optical or chemical means. Useful labels in the disclosed methods include magnetic beads (e.g., DYNABEADS®), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize the cytokine biomarkers, or secondary antibodies that recognize the antibodies to the cytokine biomarkers.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Detection methods employing immunoassays are particularly suitable for practice at the point of patient care. Such methods allow for immediate diagnosis and/or prognostic evaluation of the patient. Point of care diagnostic systems are described, e.g., in U.S. Pat. No. 6,267,722 which is incorporated herein by reference. Other immunoassay formats are also available such that an evaluation of the biological sample can be performed without having to send the sample to a laboratory for evaluation. Typically these assays are formatted as solid assays where a reagent, e.g., an antibody is used to detect the cytokine. Exemplary test devices suitable for use with immunoassays such as assays of the present methods are described, for example, in U.S. Pat. Nos. 7,189,522; 6,818,455 and 6,656,745.

### Detection of Polynucleotides

In some embodiments, this disclosure provides methods for detection of polynucleotide sequences which code for the cytokine biomarkers (e.g., CCL3, CCL4, or CXCL13) in a biological sample, e.g., for the diagnosis of a hematological malignancy. As noted above, a “biological sample” refers to a cell or population of cells or a quantity of tissue or fluid from a patient. Most often, the sample has been removed from a patient, but the term “biological sample” can also refer to cells or tissue analyzed in vivo, i.e., without removal from the patient. Typically, a “biological sample” will contain cells from the patient, but the term can also refer to noncellular biological material, such as noncellular fractions of the fluid from a subject.

### Amplification-Based Assays

In one embodiment, amplification-based assays are used to measure the level of chemokines. In such an assay, the chemokine nucleic acid sequences act as a template in an amplification reaction (e.g., Polymerase Chain Reaction, or PCR). In a quantitative amplification, the amount of amplification product will be proportional to the amount of template in the original sample. Comparison to appropriate controls provides a measure of the copy number of the cytokine biomarker associated gene. Methods of quantitative amplification are well known to those of skill in the art. Detailed protocols for quantitative PCR are provided, e.g., in Innis et al. (1990) PCR Protocols, A Guide to Methods and Applications, Academic Press, Inc. N.Y.). RT-PCR methods are well known to those of skill (see, e.g., Ausubel et al., supra). In some embodiments, quantitative RT-PCR, e.g., a TaqMan® assay, is used, thereby allowing the comparison of the level of mRNA in a sample with a control sample or value. The known nucleic acid sequences for chemokine are sufficient to enable one of skill to routinely select primers to amplify any portion of the gene. Suitable primers for amplification of specific sequences can be designed using principles well known in the art (see, e.g., Dieffenfach & Dveksler, PCR Primer: A Laboratory Manual (1995)).

In some embodiments, a TaqMan® based assay is used to quantify the cytokine biomarker-associated polynucleotides. TaqMan® based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq®, results in the cleavage of the TaqMan® probe. This cleavage separates the 5′ fluorescent dye and the 3′ quenching agent, thereby resulting in an increase in fluorescence as a function of amplification (see, for example, literature provided by Perkin-Elmer, e.g., www2.perkin-elmer.com).

In some embodiments, hybridization based assays can be used to detect the amount of CCL2, CCL3, CCL4, CCL5, CXCL13, CCL17, CCL22 and TNF-alpha in the cells of a biological sample. Such assays include dot blot analysis of RNA as well as other assays, e.g., fluorescent in situ hybridization, which is performed on samples that comprise cells. Other hybridization assays are readily available in the art.

### Formulation

The compounds of this disclosure may be formulated for administration to animal subject using commonly understood formulation techniques well known in the art. Formulations which are suitable for particular modes of administration and for the compounds of Compound A may be found in Remington's Pharmaceutical Sciences, latest edition, Mack Publishing Company, Easton, Pa.

The compounds of this disclosure may be prepared in the form of prodrugs, i.e., protected forms which release the compounds of disclosed herein after administration to the subject. Typically, the protecting groups are hydrolyzed in body fluids such as in the bloodstream thus releasing the active compound or are oxidized or reduced in vivo to release the active compound. A discussion of prodrugs is found in Smith and Williams Introduction to the Principles of Drug Design, Smith, H. J.; Wright, 2^{nd} ed., London (1988).

A compound of this disclosure can be administered as the neat chemical, but it is typically preferable to administer the compound in the form of a pharmaceutical composition or formulation. Accordingly, the present disclosure also provides pharmaceutical compositions that comprise a compound of Compound A and a biocompatible pharmaceutical carrier, adjuvant, or vehicle. The composition can include the compound of Compound A as the only active moiety or in combination with other agents, such as oligo- or polynucleotides, oligo- or polypeptides, drugs, or hormones mixed with excipient(s) or other pharmaceutically acceptable carriers. Carriers and other ingredients can be deemed pharmaceutically acceptable insofar as they are compatible with other ingredients of the formulation and not deleterious to the recipient thereof.

The pharmaceutical compositions are formulated to contain suitable pharmaceutically acceptable carriers, and can optionally comprise excipients and auxiliaries that facilitate processing of the active compounds into preparations that can be used pharmaceutically. The administration modality will generally determine the nature of the carrier. For example, formulations for parenteral administration can comprise aqueous solutions of the active compounds in water-soluble form. Carriers suitable for parenteral administration can be selected from among saline, buffered saline, dextrose, water, and other physiologically compatible solutions. Preferred carriers for parenteral administration are physiologically compatible buffers such as Hank's solution, Ringer's solution, or physiologically buffered saline. For tissue or cellular administration, penetrants appropriate to the particular barrier to be permeated are used in the formulation. Such penetrants are generally known in the art. For preparations comprising proteins, the formulation can include stabilizing materials, such as polyols (e.g., sucrose) and/or surfactants (e.g., nonionic surfactants), and the like.

Alternatively, formulations for parenteral use can comprise dispersions or suspensions of the active compounds prepared as appropriate oily injection suspensions. Suitable lipophilic solvents or vehicles include fatty oils, such as sesame oil, and synthetic fatty acid esters, such as ethyl oleate or triglycerides, or liposomes. Aqueous injection suspensions can contain substances that increase the viscosity of the suspension, such as sodium carboxy-methylcellulose, sorbitol, or dextran. Optionally, the suspension also can contain suitable stabilizers or agents that increase the solubility of the compounds to allow for the preparation of highly concentrated solutions. Aqueous polymers that provide pH-sensitive solubilization and/or sustained release of the active agent also can be used as coatings or matrix structures, e.g., methacrylic polymers, such as the Eudragit® series available from Rohm America Inc. (Piscataway, N.J.). Emulsions, e.g., oil-in-water and water-in-oil dispersions, also can be used, optionally stabilized by an emulsifying agent or dispersant (surface active materials; surfactants). Suspensions can contain suspending agents such as ethoxylated isostearyl alcohols, polyoxyethlyene sorbitol and sorbitan esters, microcrystalline cellulose, aluminum metahydroxide, bentonite, agar-agar, gum tragacanth, and mixtures thereof.

Liposomes containing the active compound of Compound A also can be employed for parenteral administration. Liposomes generally are derived from phospholipids or other lipid substances. The compositions in liposome form also can contain other ingredients, such as stabilizers, preservatives, excipients, and the like. Preferred lipids include phospholipids and phosphatidyl cholines (lecithins), both natural and synthetic. Methods of forming liposomes are known in the art. See,. e.g., Prescott (Ed.), Methods in Cell Biology, Vol. XIV, p. 33, Academic Press, New York (1976).

The pharmaceutical compositions comprising the compound of Compound A in dosages suitable for oral administration can be formulated using pharmaceutically acceptable carriers well known in the art. The preparations formulated for oral administration can be in the form of tablets, pills, capsules, cachets, dragees, lozenges, liquids, gels, syrups, slurries, elixirs, suspensions, or powders. To illustrate, pharmaceutical preparations for oral use can be obtained by combining the active compounds with a solid excipient, optionally grinding the resulting mixture, and processing the mixture of granules, after adding suitable auxiliaries if desired, to obtain tablets or dragée cores. Oral formulations can employ liquid carriers similar in type to those described for parenteral use, e.g., buffered aqueous solutions, suspensions, and the like.

Preferred oral formulations include tablets, dragees, and gelatin capsules. These preparations can contain one or excipients, which include, without limitation:

a) diluents, such as sugars, including lactose, dextrose, sucrose, mannitol, or sorbitol;

b) binders, such as magnesium aluminum silicate, starch from corn, wheat, rice, potato, etc.;

c) cellulose materials, such as methylcellulose, hydroxypropylmethyl cellulose, and sodium carboxymethylcellulose, polyvinylpyrrolidone, gums, such as gum arabic and gum tragacanth, and proteins, such as gelatin and collagen;

d) disintegrating or solubilizing agents such as cross-linked polyvinyl pyrrolidone, starches, agar, alginic acid or a salt thereof, such as sodium alginate, or effervescent compositions;

e) lubricants, such as silica, talc, stearic acid or its magnesium or calcium salt, and polyethylene glycol;

f) flavorants and sweeteners;

g) colorants or pigments, e.g., to identify the product or to characterize the quantity (dosage) of active compound; and

h) other ingredients, such as preservatives, stabilizers, swelling agents, emulsifying agents, solution promoters, salts for regulating osmotic pressure, and buffers.

In some preferred oral formulations, the pharmaceutical composition comprises at least one of the materials from group (a) above, or at least one material from group (b) above, or at least one material from group (c) above, or at least one material from group (d) above, or at least one material from group (e) above. Preferably, the composition comprises at least one material from each of two groups selected from groups (a)-(e) above.

Gelatin capsules include push-fit capsules made of gelatin, as well as soft, sealed capsules made of gelatin and a coating such as glycerol or sorbitol. Push-fit capsules can contain the active ingredient(s) mixed with fillers, binders, lubricants, and/or stabilizers, etc. In soft capsules, the active compounds can be dissolved or suspended in suitable fluids, such as fatty oils, liquid paraffin, or liquid polyethylene glycol with or without stabilizers.

Dragée cores can be provided with suitable coatings such as concentrated sugar solutions, which also can contain gum arabic, talc, polyvinyl pyrrolidone, carbopol gel, polyethylene glycol, and/or titanium dioxide, lacquer solutions, and suitable organic solvents or solvent mixtures.

The pharmaceutical composition can be provided as a salt of the active compound. Salts tend to be more soluble in aqueous or other protonic solvents than the corresponding free acid or base forms. Pharmaceutically acceptable salts are well known in the art. Compounds that contain acidic moieties can form pharmaceutically acceptable salts with suitable cations. Suitable pharmaceutically acceptable cations include, for example, alkali metal (e.g., sodium or potassium) and alkaline earth (e.g., calcium or magnesium) cations.

Compounds of structural formula (A) that contain basic moieties can form pharmaceutically acceptable acid addition salts with suitable acids. For example, Berge, et al., describe pharmaceutically acceptable salts in detail in J Pharm Sci, 66:1 (1977). The salts can be prepared in situ during the final isolation and purification of the compounds, or separately by reacting a free base function with a suitable acid.

Representative acid addition salts include, but are not limited to, acetate, adipate, alginate, citrate, aspartate, benzoate, benzenesulfonate, bisulfate, butyrate, camphorate, camphorolsulfonate, digluconate, glycerophosphate, hemisulfate, heptanoate, hexanoate, fumarate, hydrochloride, hydrobromide, hydroiodide, 2-hydroxyethanesulfonate (isothionate), lactate, maleate, methanesulfonate or sulfate, nicotinate, 2-naphthalenesulfonate, oxalate, pamoate, pectinate, persulfate, 3-phenylpropionate, picrate, pivalate, propionate, succinate, tartrate, thiocyanate, phosphate or hydrogen phosphate, glutamate, bicarbonate, p-toluenesulfonate, and undecanoate. Examples of acids that can be employed to form pharmaceutically acceptable acid addition salts include, without limitation, such inorganic acids as hydrochloric acid, hydrobromic acid, sulfuric acid, and phosphoric acid, and such organic acids as oxalic acid, maleic acid, succinic acid, and citric acid.

Basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain alkyl halides such as decyl, lauryl, myristyl, and stearyl chlorides, bromides, and iodides; arylalkyl halides such as benzyl and phenethyl bromides; and others. Products having modified solubility or dispersibility are thereby obtained.

Compositions comprising a compound of the disclosure formulated in a pharmaceutical acceptable carrier can be prepared, placed in an appropriate container, and labeled for treatment of an indicated condition. Accordingly, there also is contemplated an article of manufacture, such as a container comprising a dosage form of a compound of the disclosure and a label containing instructions for use of the compound. Kits are also contemplated under the disclosure. For example, the kit can comprise a dosage form of a pharmaceutical composition and a package insert containing instructions for use of the composition in treatment of a medical condition. In either case, conditions indicated on the label can include treatment of inflammatory disorders, cancer, etc.

### Methods of Administration

Pharmaceutical compositions comprising Compound A can be administered to the subject by any conventional method, including parenteral and enteral techniques. Parenteral administration modalities include those in which the composition is delivered by injection such as intramuscularly, intravenously, and the like. Preferably, Compound A is administered orally, as a tablet or pill, with a daily dosage of about 50-350 mg BID.

The therapeutic index of the compound of Compound A can be enhanced by modifying or derivatizing the compounds for targeted delivery to cancer cells expressing a marker that identifies the cells as such. For example, the compounds can be linked to an antibody that recognizes a marker that is selective or specific for cancer cells, so that the compounds are brought into the vicinity of the cells to exert their effects locally, as previously described (see for example, Pietersz, et al., Immunol Rev, 129:57 (1992); Trail, et al., Science, 261:212 (1993); and Rowlinson-Busza, et al., Curr Opin Oncol, 4:1142 (1992)). Tumor-directed delivery of these compounds may enhance the therapeutic benefit by, inter alia, reducing potential nonspecific toxicities that can result from radiation treatment or chemotherapy. In another embodiment, the compound of Compound A and radioisotopes or chemotherapeutic agents can be conjugated to the same anti-tumor antibody.

The following examples are offered to illustrate but not to limit the invention. In the Examples below, Compound A refers to (S)-2-(1-(9H-purin-6-ylamino)propyl)-5-fluoro-3-phenylquinazolin-4(3H)-one.

### Additional Embodiments

Embodiment 1. A method of treating a hematological disorder in a subject, comprising the steps of a) selecting a subject having an elevated concentration of at least one biomarker selected from the group consisting of CCL2, CCL3, CCL4, CCL5, CXCL13, CCL17, CCL22, and TNF-alpha; and b) administering an effective amount of a PI3K-delta inhibitor to the subject.

Embodiment 2. The method according to embodiment 1, wherein the subject has at least two elevated biomarker concentrations selected from the group consisting of CCL2, CCL3, CCL4, CCL5, CXCL13, CCL17, CCL22, and TNF-alpha.

Embodiment 3. The method according to any of the foregoing embodiments, wherein the hematological disorder is selected from the group consisting of acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), Hodgkins lymphoma, mantle cell lymphoma (MCL), follicular lymphoma, Waldenstrom's macroglobulinemia (WM), B-cell lymphoma and diffuse large B-cell lymphoma (DLBCL).

Embodiment 4. The method according to any of the foregoing embodiments, wherein at least one elevated biomarker is at least 2-fold greater than subjects free of the hematological disorder.

Embodiment 5. The method according to any of the foregoing embodiments, wherein at least one elevated biomarker is at a level above the median for the type of cancer being treated.

Embodiment 6. The method according to any of the foregoing embodiments, wherein the hematological disorder is CLL.

Embodiment 7. The method according to any of the foregoing embodiments, wherein at least one biomarker is selected from the group consisting of CCL2, CCL3, CCL4, CXCL13, and TNF-alpha.

Embodiment 8. The method according to any of the foregoing embodiments, wherein the biomarker concentration of CCL2 is greater than 750 pg/mL, CCL3 is greater than 150 pg/mL, CCL4 is greater than 250 pg/mL, CXCL13 is greater than 200 pg/mL, or TNF-alpha is greater than 50 pg/mL, or a combination of these amounts.

Embodiment 9. The method according to any of the foregoing embodiments, wherein the hematological disorder is MCL or NHL.

Embodiment 10. The method according to any of the foregoing embodiments, wherein at least one biomarker is selected from the group consisting of CCL17, CCL22, CXCL13, and TNF-alpha.

Embodiment 11. The method according to any of the foregoing embodiments, wherein the biomarker concentration of CCL17 is greater than 150 pg/mL, CCL22 is greater than 2000 pg/mL, CXCL13 is greater than 400 pg/mL, or TNF-alpha is greater than 30 pg/mL, or a combination of these amounts.

Embodiment 12. The method according to any of the foregoing embodiments, wherein the hematological disorder is NHL and the elevated biomarker is CCL17.

Embodiment 13. The method according to any of the foregoing embodiments, wherein the biomarker concentration of CCL17 is greater than 750 pg/mL.

Embodiment 14. The method according to any of the foregoing embodiments, wherein the PI3K-delta inhibitor is a compound of formula 1:

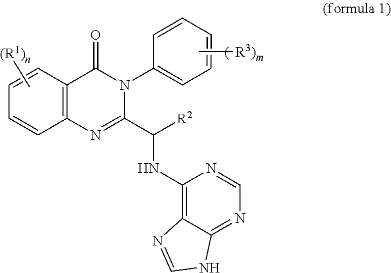

wherein each R¹ is independently selected from the group consisting of halo, CF₃, and C1-C6 alkyl; each R³ is independently selected from the group consisting of halo, CF₃, and C1-C6 alkyl; R² is hydrogen or C1-C6 alkyl; n is an integer from 0 to 2; and m is an integer from 0 to 2, or a pharmaceutically acceptable salt thereof.

Embodiment 15. The method according to any of the foregoing embodiments, wherein the PI3K-delta inhibitor is selected from the group consisting of 2-(1-(9H-purin-6-ylamino)propyl)-5-fluoro-3-phenylquinazolin-4(3H)-one; 2-(1-(9H-purin-6-ylamino)ethyl)-6-fluoro-3-phenylquinazolin-4(3H)-one; and 2-(1-(9H-purin-6-ylamino)ethyl)-3-(2,6-difluorophenyl)quinazolin-4(3H)-one or a pharmaceutically acceptable salt thereof.

Embodiment 16. The method according to any of the foregoing embodiments, wherein the concentration of at least one chemokine is decreased by at least 2-fold after administration of a PI3K-delta inhibitor.

Embodiment 17. The method according to any of the foregoing embodiments, wherein the inhibitor is administered in the amount of about 50 to 350 mg BID.

Embodiment 18. A method of predicting whether a subject with a hematological disorder will respond effectively to treatment with PI3K-delta inhibitor, comprising assessing as a biomarker in sample from the patient the amount of at least one biomarker selected from the group consisting of CCL2, CCL3, CCL4, CCL5, CXCL13, CCL17, CCL22, and TNF-alpha, and predicting the subject will respond effectively to treatment with the inhibitor.

Embodiment 19. The method according to any of the foregoing embodiments, wherein the disorder is CLL and an amount of CCL2 is greater than 750 pg/mL, an amount of CCL3 greater than 150 pg/mL, or an amount of CCL4 is greater than 250 pg/mL, or a combination of these amounts indicates that the subject is likely to respond effectively to treatment with the inhibitor.

Embodiment 20. The method according to any of the foregoing embodiments, wherein the disorder is MCL or NHL and an amount of CCL17 is greater than 150 pg/mL, an amount of CCL22 is greater than 2000 pg/mL, or an amount of CXCL13 is greater than 400 pg/mL, or a combination of these amounts indicates that the subject is likely to respond effectively to treatment with the inhibitor.

Embodiment 21. The method according to any of the foregoing embodiments, wherein the PI3K-delta inhibitor is a compound of formula 1:

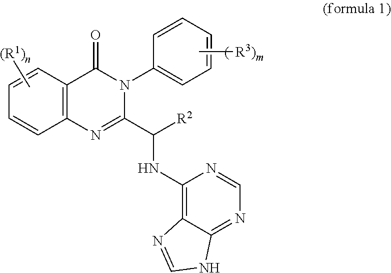

wherein each R¹ is independently selected from the group consisting of halo, CF₃, and C1-C6 alkyl; each R³ is independently selected from the group consisting of halo, CF₃, and C1-C6 alkyl; R² is hydrogen or C1-C6 alkyl; n is an integer from 0 to 2; and m is an integer from 0 to 2, or a pharmaceutically acceptable salt thereof.

Embodiment 22. The method according to any of the foregoing embodiments, wherein the inhibitor is selected from the group consisting of 2-(1-(9H-purin-6-ylamino)propyl)-5-fluoro-3-phenylquinazolin-4(3H)-one; 2-(1-(9H-purin-6-ylamino)ethyl)-6-fluoro-3-phenylquinazolin-4(3H)-one; and 2-(1-(9H-purin-6-ylamino)ethyl)-3-(2,6-difluorophenyl)quinazolin-4(3H)-one or a pharmaceutically acceptable salt thereof.

### EXAMPLE 1

### CCL3, CCL4 and CXCL13 Levels in CLL Patients Reduced After Treatment

This example provides support of Compound A reducing elevated chemokine levels in CLL patients.

Plasma samples with EDTA were collected at baseline (pre dose) and on the last day of cycle 1 (day 28) after dosing with Compound A. Samples were centrifuged at 1,100×g (relative centrifugal force) for 10 minutes at 4 degrees centigrade for separation of plasma and mononuclear cell layers. Plasma was stored at −70 degrees centigrade. Before analysis, samples were thawed overnight at 4 degrees centigrade and centrifuged at 1,500×g to remove debris. Chemokines were analyzed with commercially available multiplexed bead suspension arrays (MBA, Millipore). MBAs were analyzed using a Luminex 200 instrument and data was organized and analyzed using 3.1 xPONENT software.

The plasma concentration of 14 patients with CLL was assessed at pre-dose baseline concentrations. The average plasma concentration of CCL3 (186 pg/mL) and CCL4 (303 pg/mL) at the pre-dose baseline are approximately 5 times higher in these subject compared to normal subjects. The average plasma concentration of CXCL13 (316 pg/mL) at pre-dose baseline was approximately 10 times higher than normal subjects.

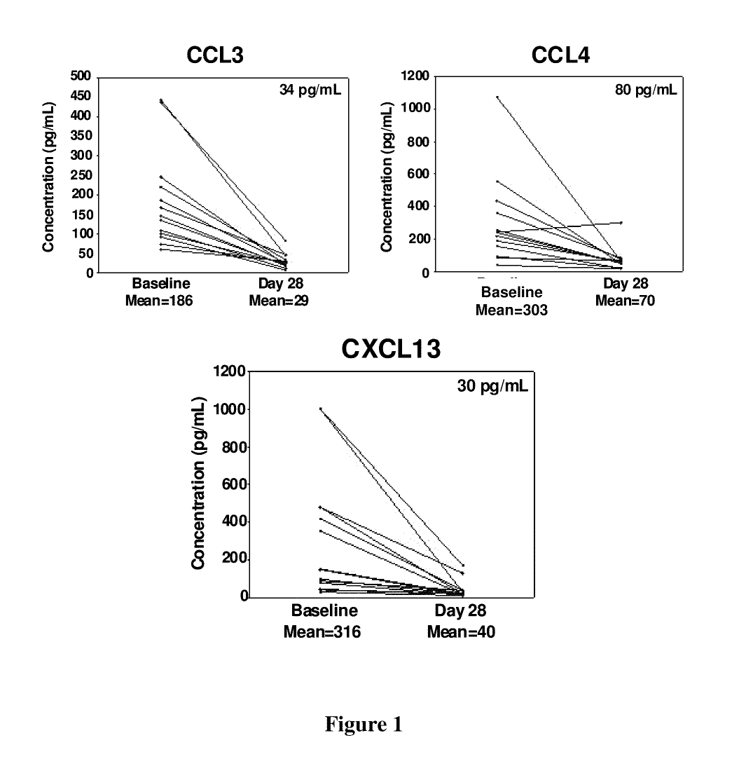

Compound A was administered at dose levels that ranged from 50 to 350 mg BID over a 28-day dosing period. In the first week of dosing across all dose levels evaluated, the concentration of CCL3, CCL4 and CXCL13 were decreased by 2 to 5-fold. On the last day of the cycle, the plasma concentration of the chemokines was reassessed, see FIG. 1. Table 1 below summarizes the chemokine concentration at the baseline and at day 28 of the cycle. A significant reduction in the average levels of CCL3, CCL4 and CXCL13 was observed after 28 days of treatment with Compound A. The reduction in chemokines was consistent with evidence of clinical activity as measured by a decrease in lymphadenopathy.

TABLE 1

| | CCL3 | CCL4 | CXCL13 |
|---|---|---|---|
| | (pg/mL) | (pg/mL) | (pg/mL) |
| Baseline | 186 | 303 | 316 |
| Day 28 | 29 | 70 | 40 |
| % reduction | 84% | 77% | 87% |

### EXAMPLE 2

### Compound A Blocks BCR-Induced Secretion of Chemokines CCL3 and CCL4 by CLL Cells

This example demonstrates that 2-(1-(9H-purin-6-ylamino)propyl)-5-fluoro-3-phenylquinazolin-4(3H)-one is effective in reducing the amount of chemokine CCL3 and CCL4 in BCR-stimulated CLL cells.

Method: CLL cells were cultured in medium (control), medium supplemented with anti-IgM or medium supplemented with anti-IgM plus Compound A. After 24 hours, supernatants were harvested and assayed by enzyme-linked immunosorbent assay and the chemokine levels compared.

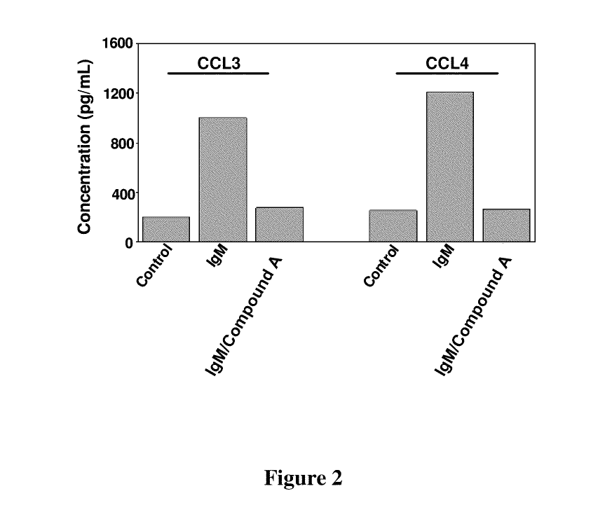

The bar diagram of FIG. 2 displays concentration of chemokine levels from CLL cells cultured in the three different conditions. Concentration of CCL3 and CCL4 were increased roughly 5 to 6 fold in the presence of anti IgM as compared to the control. Presence of Compound A, however, resulted in the effective suppression of chemokine secretion to levels nearing the control values.

### EXAMPLE 3

### Selectivity of Compound I for p110δ

This example demonstrates that Compound A is selective for p110δ as measured in isoform specific cell-based assays.

Swiss-3T3 fibroblasts and RAW-264 were seeded on a 96-well tissue culture plate and allowed to reach at least 90% confluency. Cells were starved and treated with either vehicle or serial dilutions of Compound A for 2 hrs and stimulated with PDGF or C5a respectively. Akt phosphorylation and total AKT was detected by ELISA. Purified B-cells were treated with either vehicle or serial dilutions of compound I for 30 minutes at room temperature before the addition of purified goat anti-human IgM. Results are expressed as relative [³H] thymidine incorporation induced by IgM crosslinking.

TABLE 2

| PI3Kalpha | PI3Kδ | PI3Kγ |
|---|---|---|
| EC₅₀ (nM) | EC₅₀ (nM) | EC₅₀ (nM) |
| Fibroblast Cell Line | Primary B Cell | Monocyte Cell Line |
| PDGF induced pAKT | BCR mediated | C5a induced pAKT |
| | proliferaton | |
| >20,000 | 6 | 3,894 |
| (n = 12) | (n = 6) | (n = 11) |

### EXAMPLE 4

### CCL17, CCL22, CXCL13, and TNF-Alpha Levels in MCL and iNHL Patients Reduced After Treatment

This example provides support of Compound A reducing elevated chemokine levels in mantle cell lymphoma (MCL) and iNHL patients.

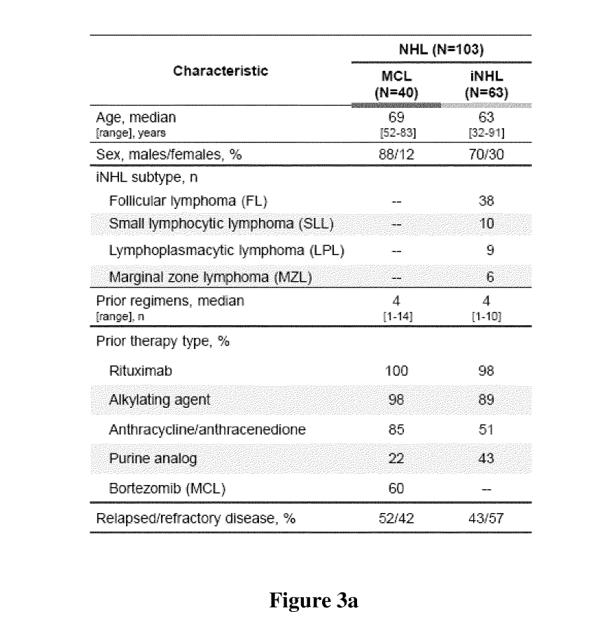

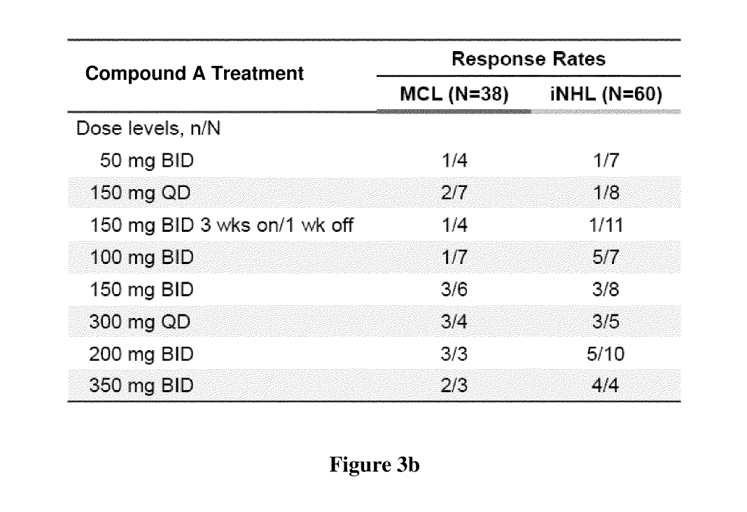

103 patients were enrolled in the study, including 40 with MCL and 63 with iNHL. Patient characteristics are summarized in FIG. 3a. Among patients with iNHL, all 4 subtypes were represented. Patients were heavily treated with chemoimmunotherapy. Patients had received a median of 4 prior regimens and some had received as many as 14 prior regimens. Most patients had received several types of chemoimmunotherapy for NHL. Disease was commonly refractory to available therapies.

Dosing of Compound A is summarized in FIG. 3b. Patients were treated over a broad range of doses of Compound A using once-per-day (QD) and twice-per-day (BID) continuous and intermittent schedules. Patients remained on therapy for protracted periods; many patients with iNHL continued on treatment beyond 12 cycles (48 weeks).

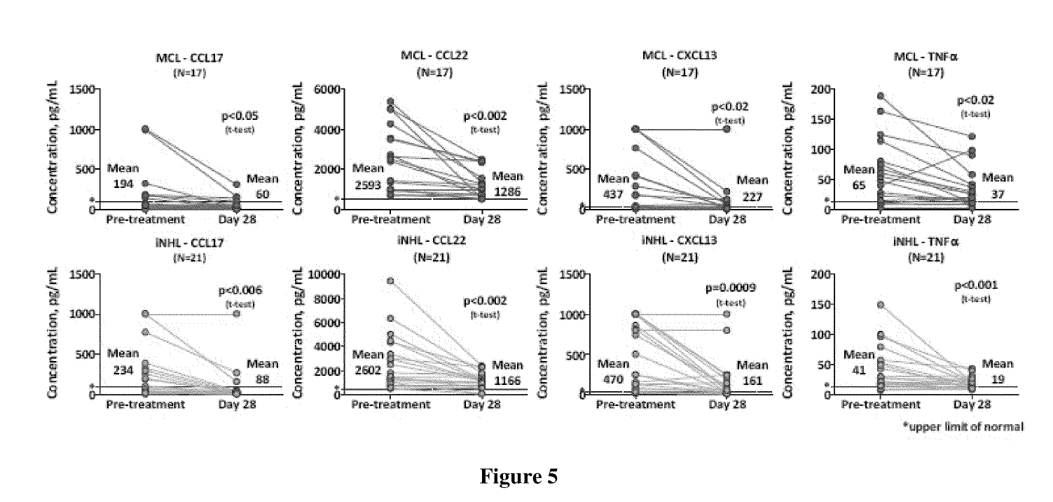

Prior to treatment, the concentration of CCL17, CCL22, CXCL13, and TNF-alpha was evaluated. On the last day of the cycle, the plasma concentration of the chemokines and TNF-alpha was re-assessed. See FIG. 5. Table 3 (for MCL) and Table 4 (for iNHL) below summarize the chemokine and TNF-alpha concentration at the baseline and at Day 28 of the cycle. A significant reduction in the average levels of CCL17, CCL22, CXCL13, and TNF-alpha was observed after 28 days of treatment with Compound A.

TABLE 3

| | CCL17 | CCL22 | CXCL13 | TNF-alpha |
|---|---|---|---|---|
| | (pg/mL) | (pg/mL) | (pg/mL) | (pg/mL) |
| Pre-Treatment | 194 | 2593 | 437 | 65 |
| Day 28 | 60 | 1286 | 227 | 37 |
| % reduction | 69% | 50% | 48% | 43% |

TABLE 4

| | CCL17 | CCL22 | CXCL13 | TNF-alpha |
|---|---|---|---|---|
| | (pg/mL) | (pg/mL) | (pg/mL) | (pg/mL) |
| Pre-Treatment | 234 | 2602 | 470 | 41 |
| Day 28 | 88 | 1166 | 161 | 19 |
| % reduction | 62% | 55% | 66% | 54% |

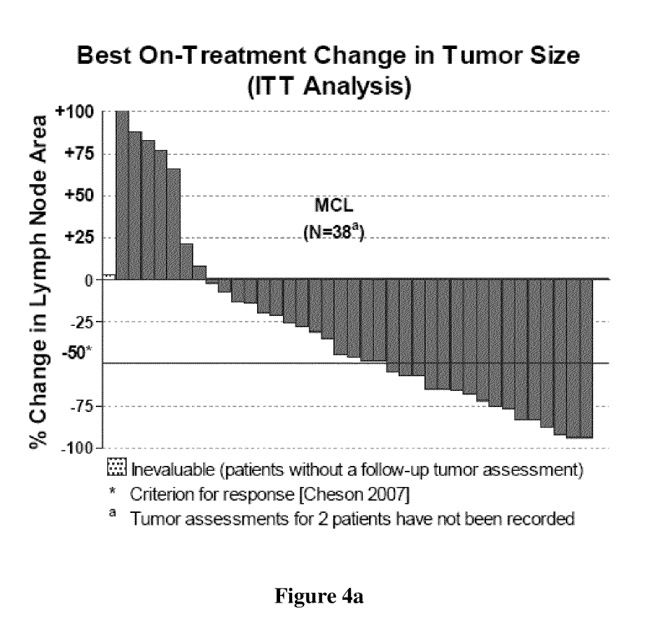

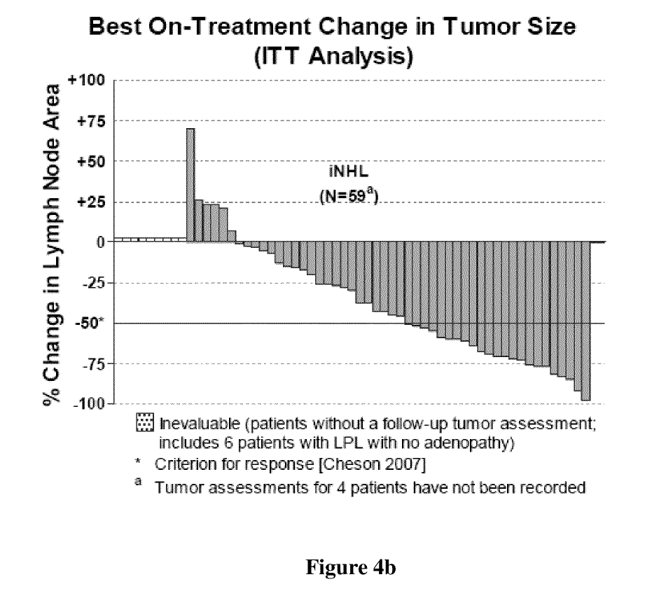

The reduction in chemokines was consistent with evidence of clinical activity as measured by the reduction in tumor size that was induced by Compound A. See FIG. 4a (MCL) and FIG. 4b (iNHL).

### EXAMPLE 5

### Plasma Cytokine and Chemokine Profiles in Patients with Hematological Malignancies

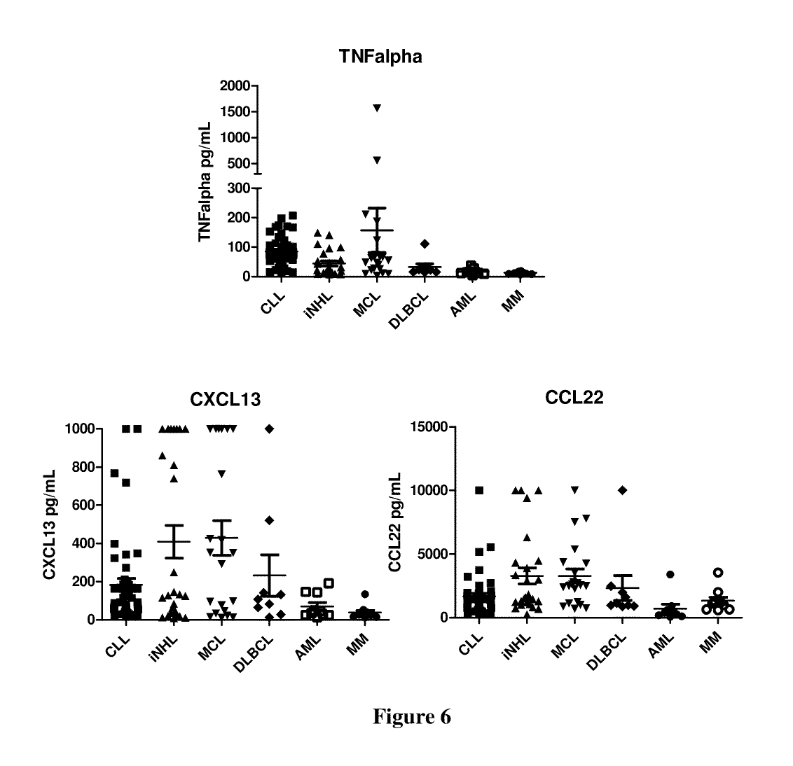

This example demonstrates that patients with certain hematological malignancies exhibit elevated levels of specific plasma cytokine and chemokines. The concentrations of CCL2, CCL3, CCL4, CCL17, CCL22, CXCL12, CXCL13, and TNF-alpha were assessed using a bead ELISA assay (LUMINEX®) of plasma samples obtained from patients with relapsed and refractory acute myelogenous leukemia (AML, n=10), chronic lymphocytic leukemia (CLL, n=50), multiple myeloma (MM, n=11), aggressive non-Hodgkins Lymphoma (aNHL, n=31) and indolent non-Hodgkins lymphoma (iNHL, n=29). Comparisons of the plasma cytokine levels were performed using a nonparametric Kruskal-Wallis statistical test followed by a Dunns post-test for group comparisons. The data is summarized in FIGS. 6, 7, and 9.

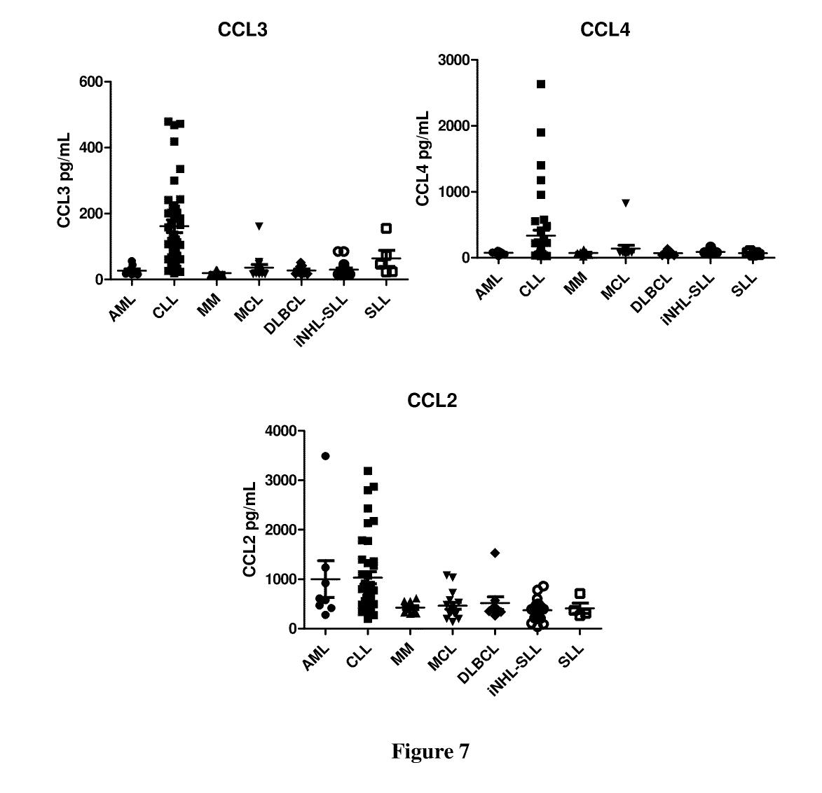

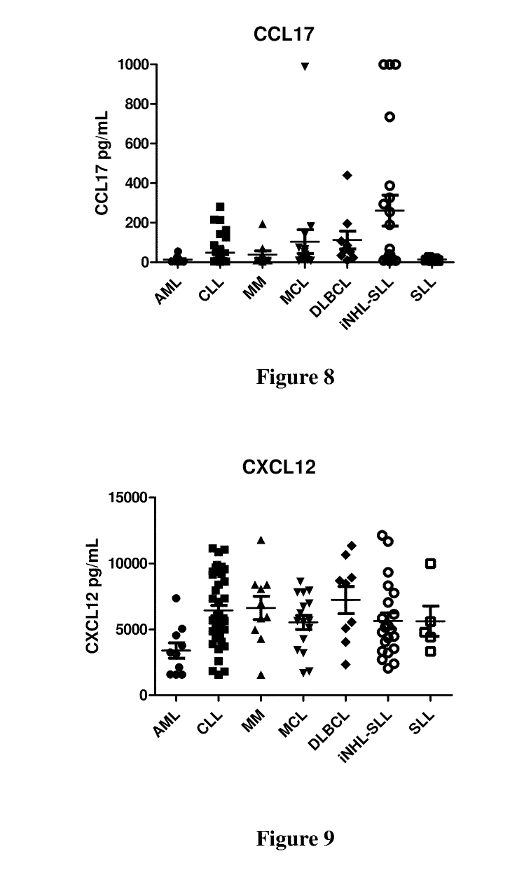

It was observed that there were >4-, >3-and 2-fold higher levels of CCL3, CCL4 (p<0.0001), and CCL2 (p<0.05), respectively, in CLL patients compared to patients with other diseases. See FIG. 7. In addition, there was a 2.5-fold higher level of CCL17 in patients with iNHL (p=0.0007) than other patients. See FIG. 8.

Thus, plasma cytokines and chemokines can serve as disease-specific inflammatory and microenvironmental factors that provide an opportunity for targeted therapeutic intervention.

### EXAMPLE 6

### Reduction of Lymph Node Size in Patients with CLL

This example demonstrates that patients with CLL having elevated plasma levels of CCL3, CCL17, and TNF-alpha experience a reduction in lymph node size when administered an effective amount of a PI3K-delta inhibitor.

In a clinical evaluation of Compound A, 2-(1-(9H-purin-6-ylamino)propyl)-5-fluoro-3-phenylquinazolin-4(3H)-one, whole blood and plasma samples from CLL patients were collected prior to administration of the PI3K-delta inhibitor, as well as after 28 days of treatment with the PI3K-delta inhibitor. The concentration of CCL3, CCL17 and TNF-alpha were determined in the plasma samples by a bead-based ELISA technology. The resulting plasma concentrations of these factors were compared to decrease in lymph node size. Patients that had lymph node decreases of 50% or greater were designated as “responders” to the drug.

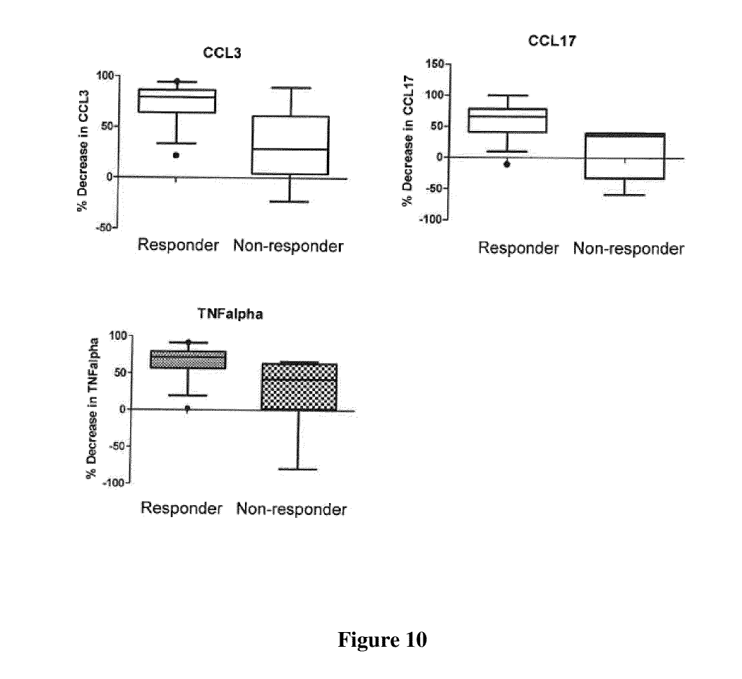

The change from predose to Day 28 for three chemokines (CCL3, CCL17 and TNF-alpha) was significantly greater for individuals that responded to the drug than those that did not, as shown in FIG. 10. Thus, the concentration change of these factors are associated with lymph node shrinkage and improved disease response.

This shows that identifying a sub-population of CLL patients having elevated CCL3, CCL17 and TNF-alpha concentrations for treatment with the PI3K-delta selective inhibitor is a useful method in providing targeted, more efficacious treatment of the disease.

1. A method of treating a hematological disorder in a subject, comprising the steps of
a) selecting a subject having an elevated concentration of at least one biomarker selected from the group consisting of CCL2, CCL3, CCL4, CCL5, CXCL13, CCL17, CCL22, and TNF-alpha; and
b) administering an effective amount of a PI3K-delta inhibitor to the subject.

2. The method according to claim 1, wherein the subject has at least two elevated biomarker concentrations selected from the group consisting of CCL2, CCL3, CCL4, CCL5, CXCL13, CCL17, CCL22, and TNF-alpha.

3. The method according to claim 1, wherein the hematological disorder is selected from the group consisting of acute lymphocytic leukemia (ALL), acute myeloid leukemia (AML), chronic lymphocytic leukemia (CLL), small lymphocytic lymphoma (SLL), multiple myeloma (MM), non-Hodgkin's lymphoma (NHL), Hodgkins lymphoma, mantle cell lymphoma (MCL), follicular lymphoma, Waldenstrom's macroglobulinemia (WM), B-cell lymphoma and diffuse large B-cell lymphoma (DLBCL).

4. The method according to claim 3, wherein at least one elevated biomarker is at least 2-fold greater than subjects free of the hematological disorder.

5. The method according to claim 3, wherein at least one elevated biomarker is at a level above the median for the type of cancer being treated.

6. The method according to claim 3, wherein the hematological disorder is CLL.

7. The method according to claim 6, wherein at least one biomarker is selected from the group consisting of CCL2, CCL3, CCL4, CXCL13, and TNF-alpha.

8. The method according to claim 7, wherein the biomarker concentration of CCL2 is greater than 750 pg/mL, CCL3 is greater than 150 pg/mL, CCL4 is greater than 250 pg/mL, CXCL13 is greater than 200 pg/mL, or TNF-alpha is greater than 50 pg/mL, or a combination of these amounts.

9. The method according to claim 3, wherein the hematological disorder is MCL or NHL.

10. The method according to claim 9, wherein at least one biomarker is selected from the group consisting of CCL17, CCL22, CXCL13, and TNF-alpha.

11. The method according to claim 10, wherein the biomarker concentration of CCL17 is greater than 150 pg/mL, CCL22 is greater than 2000 pg/mL, CXCL13 is greater than 400 pg/mL, or TNF-alpha is greater than 30 pg/mL, or a combination of these amounts.

12. The method according to claim 9, wherein the hematological disorder is NHL and the elevated biomarker is CCL17.

13. The method according to claim 12, wherein the biomarker concentration of CCL17 is greater than 750 pg/mL.

14. The method according to claim 1, wherein the PI3K-delta inhibitor is a compound of formula 1: 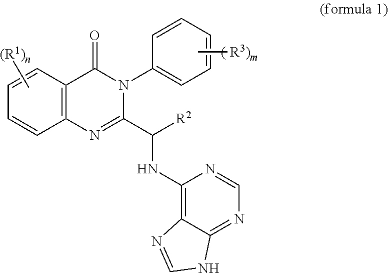
wherein each R¹ is independently selected from the group consisting of halo, CF₃, and C1-C6 alkyl;
each R³ is independently selected from the group consisting of halo, CF₃, and C1-C6 alkyl;
R² is hydrogen or C1-C6 alkyl;
n is an integer from 0 to 2; and
m is an integer from 0 to 2, or a pharmaceutically acceptable salt thereof.

15. The method according to claim 14, wherein the PI3K-delta inhibitor is selected from the group consisting of 2-(1-(9H-purin-6-ylamino)propyl)-5-fluoro-3-phenylquinazolin-4(3H)-one;
2-(1-(9H-purin-6-ylamino)ethyl)-6-fluoro-3-phenylquinazolin-4(3H)-one; and
2-(1-(9H-purin-6-ylamino)ethyl)-3-(2,6-difluorophenyl)quinazolin-4(3H)-one or a pharmaceutically acceptable salt thereof.

16. The method according to claim 1, wherein the concentration of at least one chemokine is decreased by at least 2-fold after administration of a PI3K-delta inhibitor.

17. The method according to claim 16, wherein the inhibitor is administered in the amount of about 50 to 350 mg BID.

18. A method of predicting whether a subject with a hematological disorder will respond effectively to treatment with PI3K-delta inhibitor, comprising assessing as a biomarker in sample from the patient the amount of at least one biomarker selected from the group consisting of CCL2, CCL3, CCL4, CCL5, CXCL13, CCL17, CCL22, and TNF-alpha, and predicting the subject will respond effectively to treatment with the inhibitor.

19. The method according to claim 18, wherein the disorder is CLL and an amount of CCL2 is greater than 750 pg/mL, an amount of CCL3 greater than 150 pg/mL, or an amount of CCL4 is greater than 250 pg/mL, or a combination of these amounts indicates that the subject is likely to respond effectively to treatment with the inhibitor.

20. The method according to claim 18, wherein the disorder is MCL or NHL and an amount of CCL17 is greater than 150 pg/mL, an amount of CCL22 is greater than 2000 pg/mL, or an amount of CXCL13 is greater than 400 pg/mL, or a combination of these amounts indicates that the subject is likely to respond effectively to treatment with the inhibitor.

21. The method according to claim 18, wherein the PI3K-delta inhibitor is a compound of formula 1: 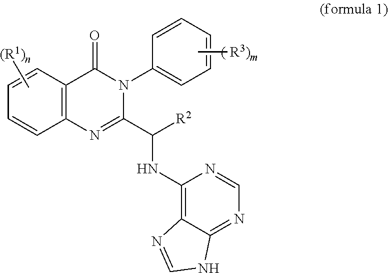
wherein each R¹ is independently selected from the group consisting of halo, CF₃, and C1-C6 alkyl;
each R³ is independently selected from the group consisting of halo, CF₃, and C1-C6 alkyl;
R² is hydrogen or C1-C6 alkyl;
n is an integer from 0 to 2; and
m is an integer from 0 to 2, or a pharmaceutically acceptable salt thereof.

22. The method according to claim 21, wherein the inhibitor is selected from the group consisting of 2-(1-(9H-purin-6-ylamino)propyl)-5-fluoro-3-phenylquinazolin-4(3H)-one;
2-(1-(9H-purin-6-ylamino)ethyl)-6-fluoro-3-phenylquinazolin-4(3H)-one; and
2-(1-(9H-purin-6-ylamino)ethyl)-3-(2,6-difluorophenyl)quinazolin-4(3H)-one or a pharmaceutically acceptable salt thereof.
